(19) United States
(12) Patent Application Publication
(10) Pub. No.: US 2007/0276311 A1
(43) Pub. Date: Nov. 29, 2007

(54) LEG WRAP

(75) Inventors: Jeffrey L. Wieringa, West Lakeland, MN (US); Michael A. Amante, St. Paul, MN (US); Maria T. Mann, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/382,397

(22) Filed: May 9, 2006

(51) Int. Cl. A61L 15/00

(52) U.S. Cl. 602/75

(57) ABSTRACT

A leg wrap is adapted to wrap around a limb of a person. The wrap includes an elongated flexible body having a first end and a second end opposite the first end along a length of the flexible body. A first fastener is positioned on the flexible body proximate the second end of the flexible body and is adapted to fasten to the flexible body. A flap is positioned between the first end and the second end of the flexible body. The flap has a first end secured to the flexible body and a second end being moveable with respect to the second body. The second fastener is positioned on a flap and adapted to fasten to the flexible body.

### BACKGROUND

Wraps, bands, braces and supports are used in a variety of different settings to reinforce and/or strengthen portions of the body. For example, these devices can be provided for joints such as knee joints and elbow joints and for limbs such as arms and legs. Current devices include a flexible material that can be wrapped around a limb and fastened using a suitable fastening mechanism. Alternatively, the devices can be formed of a tubular structure that is positioned over the limb and fastened thereto due to elastic properties of the material forming the device. These devices can be difficult to fasten to respective limbs and can be limited to use on only a particular person's body or only a particular portion of a person's body.

### SUMMARY

This summary is not intended to describe each disclosed embodiment or every implementation of the concepts presented herein. The figures and the description that follows more particularly exemplify illustrative embodiments.

In one aspect, a leg wrap is adapted to wrap around a limb of a person. The wrap includes an elongated flexible body having a first end and a second end opposite the first end along a length of the flexible body. A first fastener is positioned on the flexible body proximate the second end of the flexible body and is adapted to fasten to the flexible body. A flap is positioned between the first end and the second end of the flexible body. The flap has a first end secured to the flexible body and a second end being moveable with respect to the flexible body. The second fastener is positioned on the flap and adapted to fasten to the flexible body.

In another aspect, a method of wrapping a limb of a person is provided. The method includes providing an elongated flexible body having a first end and a second end opposite the first end along a length of the flexible body. A first fastener is provided and positioned on the flexible body proximate the second end and adapted to fasten to the flexible body. A flap is positioned between the first end and the second end of the flexible body. The flap has a first end secured to the flexible body and a second end being moveable with respect to the flexible body. A second fastener is positioned on the flap and adapted to fasten to the flexible body.

Another aspect includes a leg wrap having an elongated flexible body with a first end and second end opposite the first end along a length of the flexible body. End fastening means are positioned on the flexible body proximate the second end of the flexible body for fastening the second end to the flexible body. Intermediate fastening means positioned on a flap between the first end and the second end for fastening the flap to the flexible body.

### BRIEF DESCRIPTION OF THE DRAWINGS

The concepts presented herein will be further explained with reference to the attached figures, wherein like structure can be referred to by like reference numerals throughout the several views.

FIG. 1 is a top view of a leg wrap.

FIG. 2 is a bottom view of a leg wrap.

FIG. 3 is a sectional view as taken along lines 3-3 in FIG. 1.

FIG. 4-8 are successive views illustrating fastening a leg wrap to a person's leg.

While the above-identified figures set forth one or more embodiments of the present invention, other embodiments are also contemplated, as noted herein. In all cases, concepts presented herein describe the invention by way of representation and not by limitation. It should be understood that numerous other modifications and embodiments can be devised by those skilled in the art which fall within the scope and spirit of the principles of this invention.

### DETAILED DESCRIPTION

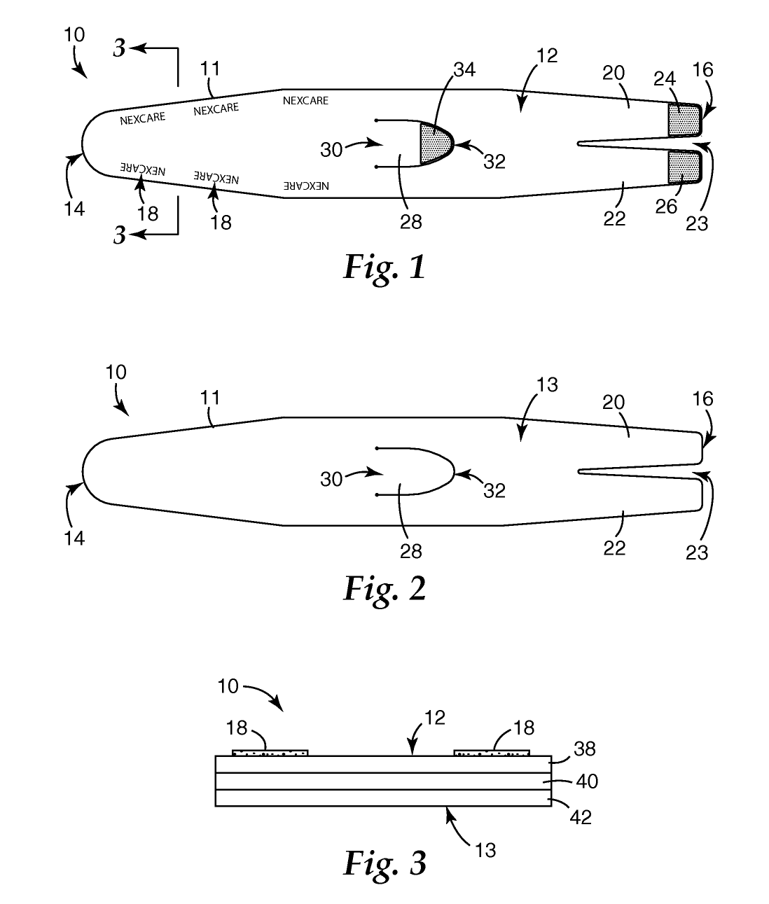

FIGS. 1-2 are plan views of a leg wrap 10 having a flexible body 11. Body 11 can be formed of a suitable, flexible material adapted to be wrapped around and fastened to a limb of the person, such as a leg. The body 11 can provide a suitable support in athletic situations as well as be configured to position and retain other components (for example therapeutic components) to the limb. The material forming body 11 can be a laminate formed of different layers of material. One example material forming body 11 is Breath-O-Prene® fabric, which is commercially available from AccuMED Technologies, Inc. of Buffalo, N.Y. This fabric is breathable to allow moisture to move away from a person's skin. Body 11 includes a first side 12 (FIG. 1) and a second side 13 (FIG. 2). In one example, side 12 includes nylon while side 13 is unbroken loop (UBL) nylon.

Leg wrap 10 has a first end 14 and a second end 16 opposite the first end 14 along a length of body 11. A plurality of raised elements 18 are positioned on side 12. The plurality of raised elements 18 can be applied to body 11 and provide a tacky or gripping surface to prevent leg wrap 10 from slipping relative to a person's leg, which can take the form of, for example, decorative indicia, user instructions and/or product identification. Leg wrap 10 further includes a first tab 20 and a second tab 22 positioned at end 16 and separated by a v-shaped gap 23. First tab 20 includes one part of a two-part mechanical fastener 24 and second tab 22 includes one part of a two-part mechanical fastener 26, where fasteners 24 and 26 are adapted to fasten to side 13 (which constitutes the other part of the two-part mechanical fastener). For example, fasteners 24 and 26 can include hooks to fasten to loops on side 13.

A flap 28 is positioned on the leg wrap 10 between first end 14 and second end 16. Flap 28 is secured to body 11 at one end 30 and includes a second end 32 that is movable with respect to flexible body 11. Flap 28 further includes one part of a two-part mechanical fastener 34 that is adapted to fasten to side 13. For example, fastener 34 can include hooks to fasten to loops on side 13. In one embodiment, flap 28 is formed by being cut out of flexible body 11. Flap 28 and fastener 34 thus provide flexibility and additional security for applying leg wrap 10 to a limb of a person.

With further reference to FIG. 3, leg wrap 10 can be formed of three layers that includes layers 38, 40 and 42. In one embodiment, layer 38 can be formed of a suitable fabric such as nylon, lycra and/or combinations thereof. This layer can be designed to be comfortable against skin of a person. Layer 40 can be formed of a polyurethane open cell foam. The open cell foam allows moisture to easily escape from side 12 against skin of a person, thus providing a comfortable situation for the person. Layer 42 can be formed of an unbroken loop (UBL) fabric that provides loops that engage fasteners 24, 26 and 34. In one embodiment, the entire side 13 includes UBL fabric, allowing increased flexibility for placement of fasteners 24, 26 and 34 onto side 13.

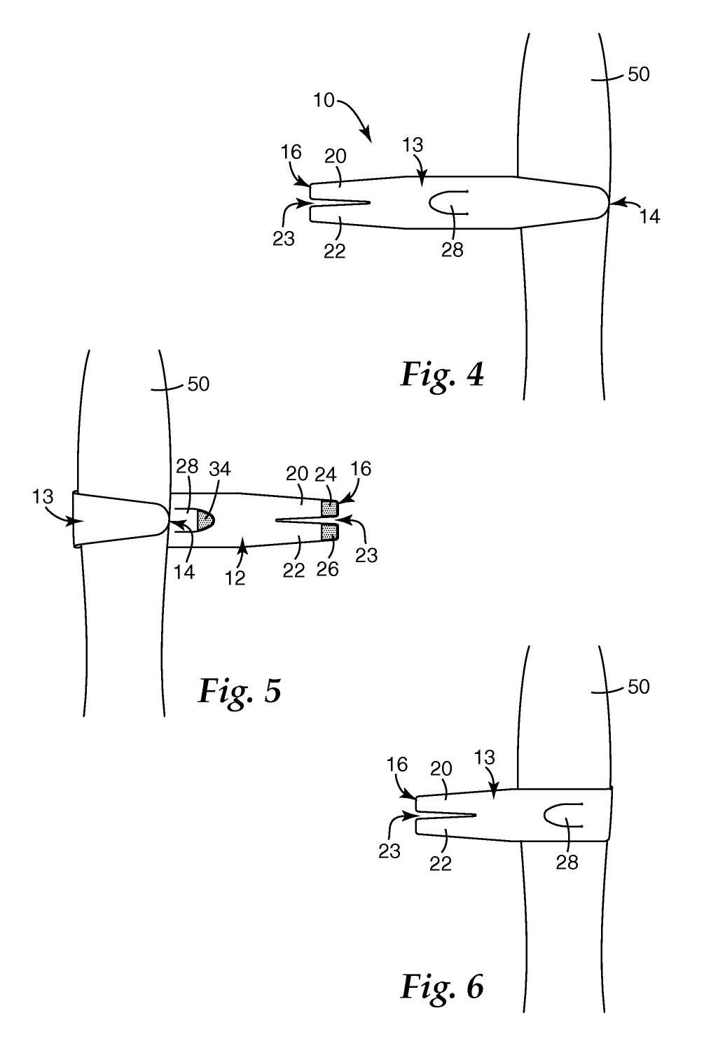

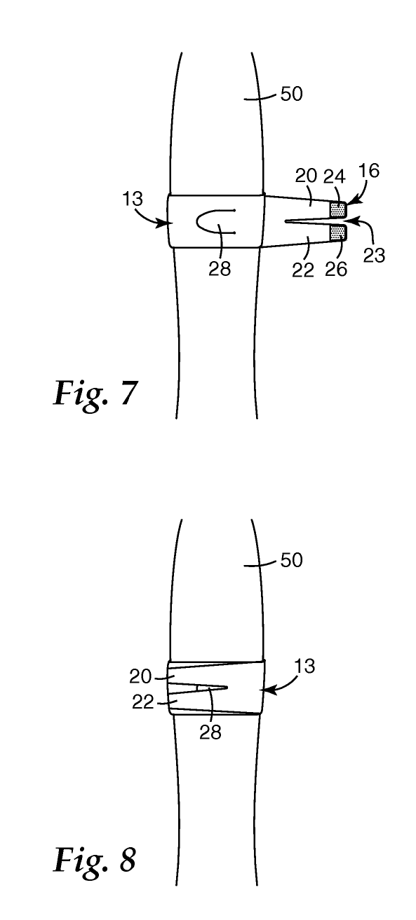

FIG. 4-8 are successive views of positioning wrap 10 around a leg 50 of a person. Wrap 10 can be positioned around a thigh of leg 50 or below a knee of leg 50. In FIG. 4, side 12, and in particular the plurality of raised elements 18, are positioned against the leg 50. As discussed above, the plurality of raised elements 18 provide a tacky or gripping surface that assists in holding body 11 to leg 50. In FIG. 5, first end 14 is held against the leg 50 while body 11 (and in particular end 16) is wrapped around the leg 50 a first time. In FIG. 6, fastener 34 is fastened to side 13 proximate first end 14. If desired, a hot or cold pack, a compression pack or other treatment aid can be applied to wrap 10 at this point to be held in place with fasteners 24 and 26. In FIG. 7, wrap 10, in particular tabs 20 and 22 are wrapped around the leg 50. In FIG. 8, fasteners 24 and 26 are secured to second side 13. As a result, a secure structure is provided around leg 50 that follows the natural form of the leg and provides excellent comfort and fit.

Although the present invention has been described with reference to several alternative embodiments, workers skilled in the art will recognize that changes may be made in form and detail without departing from the spirit and the scope of the invention. For instance, various materials and configurations of fasteners can be used. In addition, the inventive leg wrap may be used to position and retain other components relative to a person's limb to aid in patient treatment, such as a thermal pack, a compression pack, one or more magnets, a drug dispensing patch, etc. Moreover, features shown and described with respect to one embodiment may be combined with features of other embodiments, as desired.

What is claimed is:

1. A wrap adapted to wrap around a limb of a person, comprising:
an elongated flexible body having a first end and a second end opposite the first end along a length of the flexible body;
a first fastener positioned on the flexible body proximate the second end of the flexible body and adapted to fasten to the flexible body;
a flap positioned between the first end and the second end of the flexible body, the flap having a first end secured to the flexible body and a second end being movable with respect to the flexible body; and
a second fastener positioned on the flap and adapted to fasten to the flexible body.

2. The wrap of claim 1 wherein the flexible body is a laminate formed of three layers.

3. The wrap of claim 1 wherein the first fastener and the second fastener include one part of a two-part hook and loop mechanical fastener.

4. The wrap of claim 1 and further comprising a raised element having a tacky surface positioned on the flexible body.

5. The wrap of claim 1 wherein the second end includes a first tab and a second tab separated from the first tab.

6. The wrap of claim 5 wherein the first tab includes the first fastener and the second tab includes a third fastener adapted to fasten to the flexible body.

7. The wrap of claim 5 wherein the flap is spaced apart from the first tab and the second tab.

8. A method of wrapping a limb of a person, comprising:
providing an elongated flexible body having a first end and a second end opposite the first end along a length of the flexible body;
providing a first fastener positioned on the flexible body proximate the second end and adapted to fasten to the flexible body;
providing a flap positioned between the first end and the second end of the flexible body, the flap having a first end secured to the flexible body and a second end being moveable with respect to the flexible body; and
providing a second fastener positioned on the flap and adapted to fasten to the flexible body.

9. The method of claim 8 and further comprising:
positioning the first end on a limb of the person;
fastening the second fastener to the flexible body; and
fastening the first fastener to the flexible body after fastening the second fastener.

10. The method of claim 8 and further comprising:
providing a first tab in the flexible body proximate the second end, wherein the first fastener is positioned on the first tab; and
providing a second tab in the flexible body separated from the first tab, the second tab including a third fastener adapted to fasten to the flexible body.

11. The method of claim 8 and further comprising:
providing a raised tacky surface on the flexible body.

12. A leg wrap, comprising:
an elongated flexible body having a first end and second end opposite the first end along a length of the flexible body;
end fastening means positioned on the flexible body proximate the second end of the flexible body for fastening the second end to the flexible body; and
intermediate fastening means positioned on a flap between the first end and the second end for fastening the flap to the flexible body.

13. The wrap of claim 12 wherein the flexible body is formed of a laminate including three layers.

14. The wrap of claim 12 and further comprising:
a first tab positioned at the second end;
a second tab positioned at the second end and separated from the first tab.

15. The wrap of claim 14 wherein the end fastening means are positioned on the first tab and the second tab.

16. The wrap of claim 12 and further comprising:
a raised tacky surface positioned on the flexible body.

17. The wrap of claim 12 wherein the flap includes a first end secured to the flexible body and a second end that is movable with respect to the flexible body.
